(19) United States
(12) Patent Application Publication
(10) Pub. No.: US 2006/0094053 A1
(43) Pub. Date: May 4, 2006

(54) Self-assembled arrays of lipid-bilayer vesicles

(76) Inventors: Dimitrios Stamou, Copenhagen (DK); Horst Vogel, Preverenges (CH)

Correspondence Address: NIXON & VANDERHYE, PC, 901 NORTH GLEBE ROAD, 11TH FLOOR, ARLINGTON, VA, 22203, US

(21) Appl. No.: 11/266,485

(22) Filed: Nov. 4, 2005

Related U.S. Application Data
(60) Provisional application No. 60/624,757, filed on Nov. 4, 2004.

(51) Int. Cl. C12Q 1/68; C12M 1/34

(52) U.S. Cl. 435/6; 435/287.2; 977/924

(57) ABSTRACT

High-density arrays of attoliter volume elements can be created within minutes in a parallel and effortless manner by using self-assembly of nanometer-sized components (e.g., lipid vesicles containing (bio)chemicals) based on biological recognition. The ultrasmall volumes allow localization to a predefined position of a few or single molecules, and then screening for their (bio)chemical properties or performing confined (bio)chemical reactions.

### CROSS-REFERENCE TO RELATED APPLICATIONS

This application claims the benefit of provisional Appln. No. 60/624,757, filed Nov. 5, 2004; the contents of which are incorporated by reference.

### BACKGROUND OF THE INVENTION

This invention relates to processes for producing self-assembled arrays of lipid vesicles and uses thereof. Products made by the process are also provided.

Spatial compartmentalization is a prerequisite for the creation of living matter^{[1]}. Without the existence of clearly defined borders^{[2]}, differentiation and diversity at the cellular level would not be possible. Most scientific disciplines that deal with dissolved molecules are concerned with the same problem of subdividing solutions in miniaturized autonomous units, either to increase the functional complexity of a system^{[3]}, reduce reagent consumption^{[4]}, monitor fast chemical kinetics^{[5]}, or even to study single-molecules^{[6]}. We describe a method that allows the massively parallel isolation of attoliter (1 al=10⁻¹⁸ L) reaction volumes and their self-assembled positioning with 100-nm precision as an ordered array on a solid surface.

U.S. Pat. No. 4,282,287 and its reissue 31,712 disclose a multiple-layer product and a process of applying alternate, successive layers of avidin and biotin-containing extender material to a surface to modify its properties. But no self-assembly of vesicles on the surface or vessels which circumscribe a reaction volume to produce an array were taught or suggested.

U.S. Pat. No. 6,221,401 and U.S. Pat. No. 6,565,889 disclose a vesicle (“bilayer structure”) containing multiple reaction volumes (“containment units”). It is preferred therein that each vesicle is attached to another. But no self-assembly of the vesicle on a surface to produce an array was taught or suggested.

U.S. Pat. No. 6,270,983 also discloses a surface with avidin and biotin attached thereto. But reagents are then immobilized on the coated surface and reactions take place in solid phase. No self-assembly of vesicles on the surface or vessels which circumscribe a reaction volume to produce an array were taught or suggested.

U.S. Pat. No. 6,444,254 discloses microstamping a functionalized polymer surface with ligands. Avidin and biotin may be attached to the surface. No self-assembly of vesicles on the surface or vessels which circumscribe a reaction volume to produce an array were taught or suggested.

U.S. Pat. No. 6,444,318 discloses a self-assembling array, but no vesicle immobilized on the surface or vessels which circumscribe a reaction volume to produce the array were taught or suggested.

U.S. Pat. No. 6,855,329 discloses a patterned surface with ligands attached by a biotin-avidin-biotin linkages. But no self-assembly of vesicles on the surface or vessels which circumscribe a reaction volume to produce an array were taught or suggested.

WO 00/73798 discloses functionalized, polymer-reinforced (sterically stabilized) vesicles, but they are not self-assembled to produce an array.

US 2004/0241748 A1 discloses self-assembling arrays, but they are different from the arrays of the present invention.

US 2005/0019836 A1 and WO 02/046766 disclose vesicles which circumscribe a reaction vesicle, but they are also different from the arrays of the present invention.

The present invention is directed to an improved self-assembled array of lipid vesicles, processes for producing them, and processes for using them to address problems of the art. Other advantages and improvements are described below or would be apparent from the disclosure herein.

### SUMMARY OF THE INVENTION

It is an object of the invention to self assemble an array comprising a surface (or substrate) and lipid vesicles immobilized thereon.

In one embodiment, an array may comprise (i) a surface and receptors attached thereto in a pattern and (ii) vesicles having a lipid bilayer, ligands exposed on the vesicle's exterior, and chemical reagents or proteins contained by the vesicle (i.e., encapsulated in the vesicle's aqueous interior or embedded in the lipid matrix). A vesicle may be located in one or more region(s) on the surface by specific binding between ligand exposed on the vesicle's exterior and receptor attached to the region(s). Areas, which do not have receptor attached thereto, separate regions from each other on the surface. The lipid bilayer of a vesicle is comprised of charged lipids, uncharged lipids, and hydrophilic modified lipids.

At least 10⁴ regions, at least 10⁵ regions, at least 10⁶ regions, at least 10⁷ regions, or at least 10⁸ regions per mm² may be formed on the surface. Each region may be separated from its nearest neighboring region by a center-to-center distance of at least 1 μm, at least 10 μm, at least 100 μm, at most 1 μm, at most 10 μm, at most 100 μm, or any combination thereof. The surface may be glass, metal conductor or metal oxide semiconductor, nonporous film or porous membrane (e.g., polyester, nylon), or polymeric material (e.g., polyacetate, polycarbonate, polystyrene).

There may be about one vesicle immobilized in each region (e.g., average number of vesicles in each region is from 0.5 to 10). For single vesicle occupancy, immobilization may be performed with a dilute concentration of vesicles. It is preferred that at least 50%, at least 60%, at least 70%, at least 80%, or at least 90% of all regions are occupied by one or more vesicles. The average diameter of the vesicles may be at least 10 nm, at least 50 nm, at least 100 nm, at least 500 nm, at least 1 μm, at most 50 nm, at most 100 nm, at most 500 nm, at most 1 μm, at most 10 μm, at most 100 μm, or any combination thereof. Small vesicles are preferred for single vesicle reactions. Vesicles may be made by electroformation, extrusion, or sonication. Vesicles of substantially uniform size are preferably made by extrusion, but they may also be selected by sizing through a membrane or gel exclusion; their use provides a substantially equal density of vesicles across each region or the entire surface.

The “pattern” may be regular or irregular with respect to a repeating arrangement of receptor-attached regions where at least one vesicle is immobilized on the surface. Examples of a regular pattern are tiling (or tessalation) of regions which cover the surface with no gaps or overlaps, and regions with rotational and/or translational symmetry which allow gaps while covering the surface. An irregular pattern is characterized by the absence of any discernible repeating arrangment of regions. Each region may be elliptical (e.g., circle) or polygonal (e.g., rectangle) on the two-dimensional surface, and separated from each other by areas where receptors are not immobilized. Thus, receptors are attached to the surface in each “region” but not in the “area” separating regions. Tiled regions may be separated from each other by areas along the region's border; symmetric regions may be separated from each other by areas which are the gaps between the regions.

Receptor and ligand may be streptavidin and biotin, respectively, or analogs thereof (e.g., monomeric avidin or streptavidin, and DSB-X biotin). Alternatively, they may be antibody binding site/hapten, complementary oligonucleotides, polyhistidine/alkaline earth metal, or electrostatic interactions between a positively-charged surface and a negatively-charged vesicle, respectively. It is preferred that the ligand-conjugated lipids be about 2% of all lipids of the vesicle (e.g., from 1 mol % to 5 mol %).

Negatively-charged (anionic) lipids of the vesicle include: cardiolipin, diacyl-glycero-phosphatidic acid, diacyl-phosphatidylglycerol, diacyl-phosphatidylinositol, and diacyl-glycero-phosphatidylserine. Positively-charged (cationic) lipids are not preferred for making the vesicle. Uncharged lipids of the vesicle include: cholesterol (in mixtures with other lipids), diacyl-phosphatidylethanolamine, and sphingomyelin. Archae lipids can be included for their chemical and mechanical stability. It is preferred that the charged lipids be about 10% of all lipids of the vesicle (e.g., from 8 mol % to 15 mol %).

The lipid bilayer may be modified with a hydrophilic polymeric chain (e.g., a PEG) or glycolipid (e.g., ganglioside GM₁) to sterically stabilize the vesicle. It is preferred that the modified lipids be about 0.3% of all lipids of the vesicle (e.g., from 0.1 mol % to 1 mol %).

It is another object of the invention to provide a process for producing the array. The arrays and intermediate products made during the production process may then be subjected to further processing and/or use. It is yet another object of the invention to provide a process for using the array.

Arrays may be produced by attaching receptors to regions of the array where vesicles will be immobilized, but not to areas of the array where vesicles will not be immobilized. Vesicles are made from charged lipids, uncharged lipids, and hydrophilic modified lipids; the lipid bilayer spatially compartmentalizes the interior from the exterior of each vesicle, and ligand is exposed the exterior of vesicles. Vesicles are immobilized on the array by specific receptor-ligand binding and located at “regions” instead of “areas” of the surface.

Receptors may be patterned on the surface by contact printing (see Xia & Whitesides, Angew. Chem. Int. Ed. 37:550-575, 1998), electron beam lithography (see Stamou et al., Langmuir 20:3495-3497, 2004), or dip pen lithography using cantilevers of atomic force microscopy. Successive steps of immobilizing vesicles may be used to “activate” different regions of the surface by attaching receptors, binding ligand-bearing vesicles to receptors, and blocking unbound receptors with an excess of free ligands. Different vesicles can be localized to particular regions using a single receptor-ligand pair in many separate self-assembly steps. Alternatively, several receptor-ligand pairs (e.g., complementary oligonucleotides) may be used to “activate” different regions of the surface by attaching all of the receptors at the same time. Different vesicles can be localized to particular regions in the same self-assembly step. “Three-dimensional” patterns may be produced by building up layers through successive steps of constructing planar vesicle structures. Receptor may be attached to a region through a ligand which was previously fixed on the surface; this ligand may be the same or different from the vesicle-borne ligand.

Chemical reactions may be controlled by segregating reagents in separate vesicles, and then initiating the reaction by disrupting the lipid bilayers and/or mixing the contents of one or more vesicles. Vesicles may be released from the surface by breaking the receptor-ligand interactions (e.g., monomeric stretavidin and/or DSB-X) under mild conditions without lysing vesicles or denaturing their contents. Such elution requires a reversible receptor-ligand interaction, but covalent crosslinking may be used if reversible binding is not required. Selective release of vesicles from the surface by eluting with excess free ligand, may then be followed by chemical analysis of free vesicles and/or empty surface by an analytical technique (e.g., infrared, Raman, or mass spectroscopy). A combinatorial library comprising chemical compounds or enzymes may be immobilized in vesicles of an array, then screened for chemical reactivity or enzyme activity (e.g., hydrolysis, transferase).

Processes for using and making arrays are provided. It should be noted, however, that a claim directed to the product is not necessarily limited to a process unless the particular steps of the process are recited in the product claim.

Further aspects of the invention will be apparent to a person skilled in the art from the following description of specific embodiments and the claims, and generalizations thereto.

### BRIEF DESCRIPTION OF THE DRAWINGS

FIG. 1 illustrates a strategy to self-assemble SVs on a surface with spatial control. First, biotinylated bovine serum albumin (BSA-biot) is fixed on the surface in a defined pattern by microcontact printing (μCP). The nonprinted areas are blocked by adsorbtion of BSA from solution. Streptavidin (Strept) is then bound to the printed BSA-biotin. Biotinylated lipids mediate the specific immobilization of vesicles. The vesicles carry charged and poly(ethylene glycol) (PEG)-derivatized lipids to prevent nonspecific interactions with the surface: 1-palmitoyl-2-oleoyl-sn-glycero-3-phosphocholine (POPC); 1-palmitoyl-2-oleoyl-sn-glycero-3-phospho-rac-(1-glycerol) (POPG); n-((6-(biotinoyl)amino)hexanoyl)-1,2-dihexa-decanoyl-sn-glycero-3-phosphoethanolamine (DHPE-biotin); and 1,2-dioleoyl-sn-glycero-3-phosphoethanolamine-n-[methoxy(polyethylene glycol)-2000] (DOPE-PEG₂₀₀₀). The triangle and pentagon represent different water-soluble molecules confined in the vesicle interior.

FIGS. 2A-2B show confocal fluorescence microscopy characterization of groups of vesicles arrayed on a glass surface (LSM 510, Zeiss). Binding sites were 2 μm in diameter and situated on an 8 μm two-dimensional lattice. Vesicles were labeled with 1% rhodamine-lipid in the bilayer and loaded with 200 mM D-sorbitol and 100 μM carboxyfluorescein (about 30 CF molecules per 100 nm vesicle). FIG. 2A shows fluorescence from the lipid bilayer, which indicates the position of the vesicles. FIG. 2B shows a line trace from the image in FIG. 2A showing the rhodamine signal (in red) and the simultaneously acquired fluorescence of CF (in green). The vesicles are positioned site-specifically on the surface and remain intact as demonstrated by their retention of CF.

FIGS. 3A-3C show high-density arrays of SVs immobilized on 100 nm×400 nm binding sites, separated by 800 nm. The confocal fluorescence images (red: rhodamine; green: Oregon 488) reveal: an array of one type of vesicle labeled with Oregon 488-lipid (FIG. 3A) and the same patterns incubated with a mixture of two differently-labeled vesicle populations (FIG. 3B). Overlay of the two images shows no co-localization of fluorescence, which proves that only one vesicle was immobilized per binding site. FIG. 3C shows that directed SA permits the construction of complex high-density SV arrays over large areas.

FIG. 4 shows the internal pH of SVs monitored over time. Vesicles were loaded with 100 μM carboxyfluorescein (CF) dye. The pH value was inferred from the excitation ratio 488 nm/458 nm of the dye, at an emission bandwidth of 505 nm to 550 nm. Firstly, the pH value of SVs was demonstrated to be stable after their immobilization (open gray markers). Secondly, a chemical reaction (deprotonation of CF) was triggered in the interior of the SVs (solid black markers) by the addition of 10 nM gramicidin A (Gram). In both, vesicles 0.5 μm to 1 μm in diameter were selected for use to ensure a sufficient number of fluorophores were present. For clarity, the results of the first example are shifted vertically in the graph by −0.1 unit in the pH value.

FIG. 5 shows the calibration curve which was calculated for two images at 458 nm and 488 nm, respectively, for each time point in FIG. 4. The intensity of each vesicle was noted in each of the two images to calculate the corresponding ratio value for a vesicle at a given time. pK of carboxyfluoroscein (CF, closed squares) is 6.12±0.02 and pK of 2′,7′-bis-(2-carboxyethyl)-5-(and-6)-carboxyfluorescein (BCECF, open squares) is 7.05±0.02.

### DESCRIPTION OF SPECIFIC EMBODIMENTS OF THE INVENTION

By using lipid-bilayer vesicles^{[1]} as molecular shuttles^{[7]}, we transported and localized (bio)molecules encapsulated in their aqueous interior or embedded in the lipid matrix. The site-selective immobilization of intact single vesicles (SVs) was mediated by patterns of receptor molecules defined by microcontact printing (μCP)^{[8]} on glass. One-step directed self-assembly (SA)^{[9]} produced arrays of about 10⁶ volume-elements per mm² within minutes. As illustrated, this approach can additionally create random arrays of vesicles cof varied content that may serve as libraries of miniaturized (bio)chemical reaction systems.

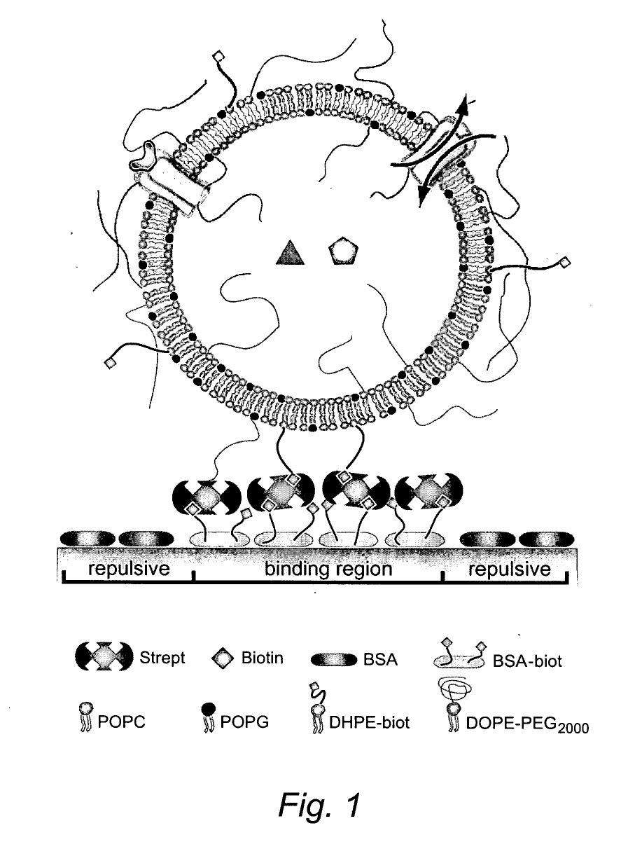

The strategy employed to construct arrays of surface-immobilized SVs is illustrated in FIG. 1. Similar concepts have been recently applied to the patterned immobilization of colloids^{[1]} or vesicles^{[11]}. We defined regions on the surface that specifically bind vesicles and are surrounded by areas that prevent nonspecific attachment. Specific binding is mediated through the receptor-ligand pair of streptavidin-biotin^{[12]}. Avidin may be used instead of streptavidin. To break the receptor-ligand interaction under mild conditions, monomeric streptavidin/avidin and/or desthiobiotin (DSB-X) may be used. Another receptor-ligand pair that may be used is an antibody binding site and a hapten (e.g., digoxygenin). Lipid-bilayer vesicles with exposed biotin ligands on their outer leaflet will specifically bind to free binding sites of the surface's regions where streptavidin receptors are attached in a pattern^{[13]}. In this manner, the positioning of vesicles and their content becomes a diffusion-limited SA process guided by the patterned surface functionalization.

The properties of vesicles can be tailored for optimum interaction with the surface by selecting an appropriate lipid composition for their bilayer. See WO 00/73798, the contents of which are incorporated by references herein.

We adjusted the lipid composition to introduce two long-range repulsive forces that prevent nonspecific interactions between the vesicles and the surface. Electrostatic repulsion was controlled by setting the ratio of charged to uncharged lipids. The presence of 10% charged lipids (e.g., 1-palmitoyl-2-oleoyl-sn-glycero-3-phospho-rac-(1-glycerol) or POPG) also increases the bending energy^{[14]} of the lipid membrane, which prevents vesicles from deforming and fusing upon immobilization. To establish a second barrier against intimate contact between the surface and vesicles, we employed lipids modified with a hydrophilic polymeric chain (e.g., poly(ethylene glycol) or PEG)^{[15]}. The PEG molecules induce a force that is entropic in nature and independent of the immobilization conditions (e.g., pH, ionic strength, etc.) that would otherwise affect the electrostatic-based repulsion. To maintain the membrane in a fluid state, the main lipid constituent (e.g., 1-palmitoyl-2-oleoyl-sn-glycero-3-phosphocholine or POPC) was chosen to have an ordered-to-fluid phase transition far below room temperature. Other lipid compositions and processes for producing vesicles are described in WO 00/73798, the contents of which are incorporated by references herein.

Balancing the contributions of the three most important interactions (i.e., receptor-ligand binding, electrostatic repulsion, and entropic force) was crucial to achieving selective deposition of intact vesicles. This may be done empirically by starting from initial conditions such as those exemplified herein.

We chose the dimensions of the vesicles and the resolution of the patterning method to be complementary to the probe volume of fluorescence-based detection techniques. Most of the vesicles used here were prepared with an average diameter of 100 nm using extrusion^{[16]} The surface onto which the vesicles are immobilized was structured by μCP, a versatile technique to pattern surfaces with a variety of receptors^{[17]} and a resolution of less than 100 nm^{[18]}. In this patterning step, we directly defined both the geometry and the resolution of the subsequent process of vesicle assembly on the solid surface.

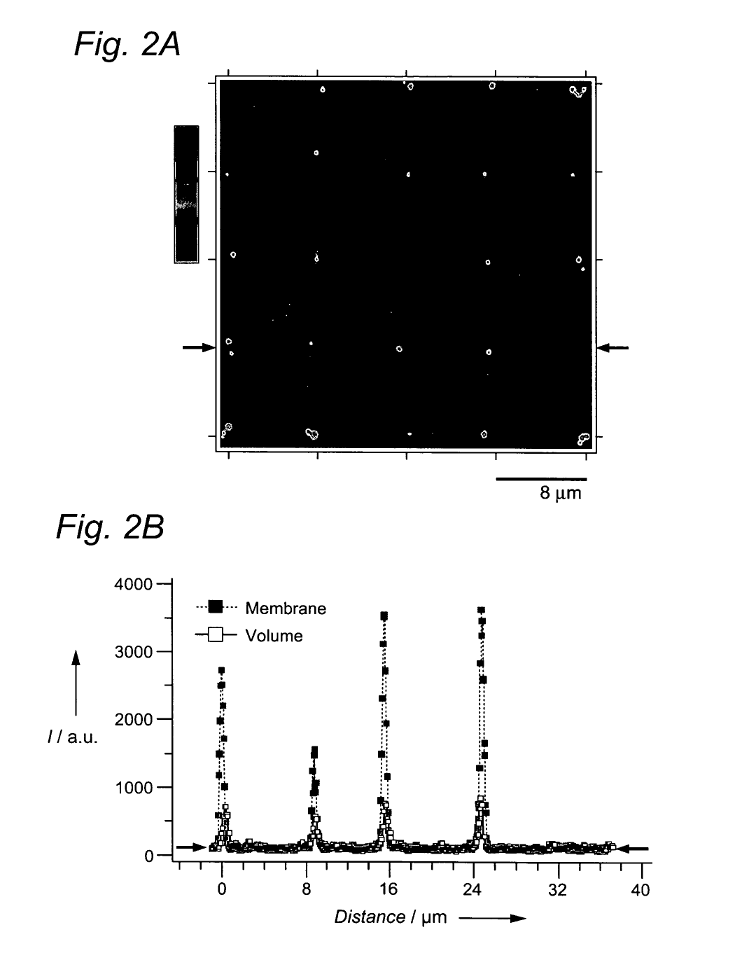

One strategy to immobilize small numbers of vesicles onto predefined regions of a surface is to reduce the number of vesicles available for binding per printed region. This is readily accomplished in the SA step by reducing the vesicle concentration. The fluorescence image in FIG. 2A shows vesicles with an average diameter of 100 nm immobilized on an array composed of 2 μm-wide dots separated by 8 μm. The vesicles were localized within ±1 μm of the center of the printed regions. Vesicles did not bind to surfaces preincubated with biotin (results not shown), which emphasizes the specificity of this immobilization procedure and the successful suppression of nonspecific interactions. The occupation of potential binding regions was on the order of 80%. The mean number of vesicles per dot is estimated to be 1.3 (all resolved features were assumed to be SVs under these dilute concentrations). To verify that vesicles were neither leaking nor fusing with the surface upon immobilization, we loaded them with the water-soluble dye carboxyfluorescein (CF). FIG. 2B shows the simultaneous recorded fluorescence emission originating from the membrane and from the interior of the vesicle, as well as the co-localization of the two signals. CF was retained for periods of several days, which proves that the immobilized vesicles had preserved their ability to confine molecules that is comparable to that of vesicles in suspension. As long as equiosmolar solutions were used, immobilized vesicles were stable against washes/buffer exchange. The absence of nonspecific deposition of vesicles together with the precise localization of immobilized vesicles create the contrast that is key for proceeding to the immobilization of single vesicles.

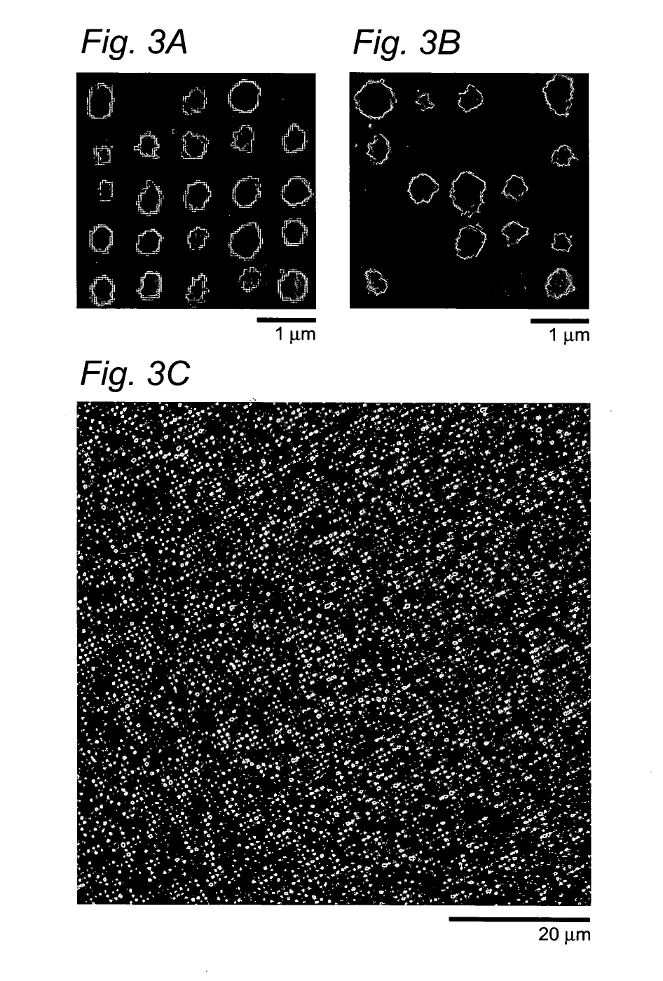

Extension of the technique from statistical placement of small numbers of vesicles on predefined region to SV positioning required reduction of the pattern-size to the dimension of the vesicles^{[10]}. We functionalized glass using high-resolution μCP stamps that had various features with sizes as small as 60 nm^{[18]}. The simultaneous presence of different patterns on the surface allowed us to screen in one step the geometries and sizes that were appropriate for having a single immobilized vesicle on a printed feature. The highest occupation of printed sites by SVs, 80%, was obtained for “linelets” having dimensions of 100×400 nm² spaced 800 nm apart (FIG. 3A). Vacancies (e.g., first row, positions 2 and 5 in FIG. 3A) are due to patterning defects (e.g., incomplete stamping) and size-related binding constraints (e.g., the number of receptors available). Decreasing the size of the patterns lowered the occupation percentage, whereas increasing it resulted in multiple occupation (results not shown). On the linelets, vesicles are localized within ±50 nm in the vertical and ±200 nm in the horizontal direction. They are kept at an average separation distance of 800 nm, which is twice their resolution-limited diameter. The different intensities observed probably originate from differences in size and, therefore, different numbers of fluorophores per vesicle. Incubation time to self-assemble vesicles on printed sites was 5 min to 10 min for a vesicle concentration of 3 nM.

In addition, we incubated the linelet patterns with a mixture of two vesicle populations, each tagged with a different fluorophore (FIG. 3B). The occupation of one site by multiple vesicles would result in the co-localization of the two fluorescence signals. No co-localization was observed, which proves that each occupied site of the pattern has only one vesicle. The size of the arrays we fabricated was 0.4×0.4 mm² and this size can be extended into the centimeter range. The fluorescence image in FIG. 3C shows a small part of a mixed array with an SV density of about 10⁶ per mm². All SVs in the image are placed in an ordered fashion on the surface, which renders their localization simple. The positioning of different SVs on the array is random, but various encoding schemes (e.g., oligonucleotide tagging) can be employed to ascertain their identity, even in the case of complex mixtures^{[19]}.

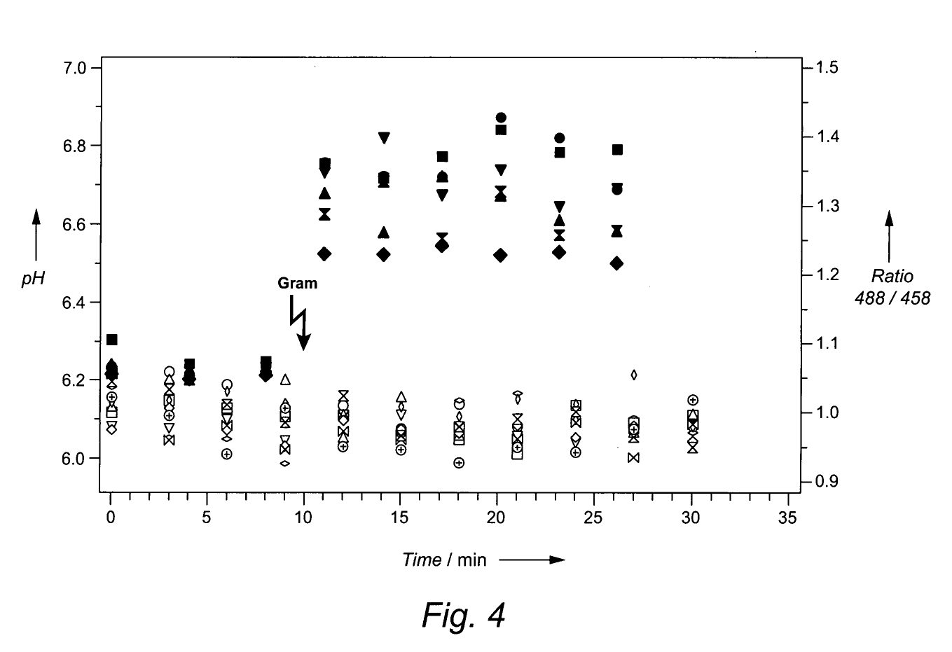

To assess the stability of the controlled environment of molecules confined inside immobilized vesicles, we investigated the permeability of the lipid bilayer. Low passive influx/efflux of ions into/out of vesicles is a prerequisite for keeping the pH or ionic composition of an SV stable, and the function of a protein or the reactivity of solutes inside an SV reliable. The pH sensitivity of CF was used to monitor the pH inside SVs, which are 0.5 μm to 1 μm in diameter, immobilized on 10 μm wide stripes. The vesicles were prepared to have a pH of about 5.5 and were immobilized in a buffer of pH 7.4. After immobilization, the pH value of the vesicles shifted to about 6.2, where it remained stable because of the established diffusion potential (FIG. 4). The SVs proved capable of maintaining pH gradients of more than one unit over the course of several hours as a result of the low counterion exchange rate.

Subsequently, we addressed the interior of these tightly sealed containers and initiated a simple chemical reaction. The deprotonation of CF loaded in SVs was triggered using an ion channel-forming peptide (gramicidin A). Gramicidin A forms cation-selective channels in lipid membranes. Upon incorporation into the vesicle's lipid bilayer, the channel allows Na⁺ ions to diffuse down their concentration gradient, in this case into the vesicle. The sodium influx induces a pH increase (proton efflux) to maintain electroneutrality. Individual vesicles were monitored over time and how the fluorophores trapped in their interior respond to the gramicidin A stimulus was recorded (FIG. 4). Due to the high single-channel conductance of gramicidin A in the lipid bilayer (˜40 pS), equilibrium was reached milliseconds after the first channel opened^{[20]}, much faster than the time resolution shown in FIG. 4. Ion channels with smaller conductivities (e.g., the 5-HT_{3A} receptor^{[21]}) would need seconds or minutes to establish equilibrium, and would therefore potentially allowing real-time monitoring of channel activity^{[22]}. This illustrates how the selective permeability properties of membrane associated transporters or channels can be employed to perform chemistry inside individually addressed nanocontainers.

Alternatively, the immobilized vesicle may be targeted by a vesicle in solution using a receptor-ligand interaction, and then fusion of the vesicles and mixing of their contents may be triggered through SNAREpins. SNARE-mediated fusion between vesicles will immobilize them on a surface, mix their contents, initiate a chemical reaction using any reagents that might be present, and contain any reaction product(s) within the fused vesicle. The product(s) may be released from the surface by lysing the lipid bilayer and/or breaking the receptor-ligand interaction. Only chemicals that are transformed in the reaction are considered “reagents” herein, in contrast to inert chemicals like the aqueous solution, salts, and buffer. The specificity of v- and t-SNARE interactions may be used to combinatorially vary the pairing of vesicles with different contents by faithful targeting of vesicles with complementtary SNAREs, and then fusing their lipid bilayers through SNAREpins.

Combinatorial libraries of chemical compounds or enzymes may be arrayed. A unique or a few different compound(s)/enzyme(s) may be enclosed in each vesicle, which is then immobilized. The same region on each array may be activated by attaching the receptor, at least one vesicle which contains the unique or the few different compound(s)/enzyme(s) of the combinatorial library may be immobilized on the surface through ligand on the vesicle's exterior leaflet, free receptors are blocked with an excess of ligand in solution, and the cycle is repeated on another region on each array for another unique or few different compound(s)/enzyme(s). Here, compounds/enzymes are identified by their location on the array; alternatively, each vesicle could contain an nucleic acid tag in the vesicle's aqueous interior or lipid matrix and the compound/enzyme is identified by the tag's nucleotide sequence.

Hierarchical SA is an emerging approach to the fabrication of functional nano-sized architectures^{[9]}. Here, SA principles and receptor-ligand interactions are combined to define attoliter autonomous reaction volumes, and then to order them on a surface. Incorporation of multifunctional recognition elements, like oligonucleotides, would further increase the complexity of the assembled structures^{[23]}. The lipid-bilayer vesicles we used as molecular vessels are arrayed at high densities (every 800 nm, about 10⁶ per mm²). Nevertheless, each one maintains its cargo dissolved in a protective environment^{[24]} of defined chemical composition (pH, ionic strength, etc.) and at the same time localizes its position with 100-nm precision. Such ultra-small volume libraries allow simultaneous screening^{[6]} of (bio)chemical properties, molecular function, or confined chemical reactions over millions of samples, while scarce reagents are conserved (for a total reaction volume of a few picoliters). A natural extension of this work is the use of vesicles produced directly from cells to form arrays^{[25]}. To array native vesicles individually, the employment of a more versatile surface modification^{[26]} might be crucial since their properties are not so easily controlled as those of synthetic vesicles. Different enzymes may be encapsulated in the vesicle's aqueous interior or embedded in its lipid matrix, and then arrayed. Native vesicles are of primary importance as they can carry receptor proteins expressed in cell membranes and/or signal transduction machinery from the cytosol. Alternatively, functional membrane proteins (e.g., ion channels or pumps, receptors, signal transduction machinery, SNAREs, transporters) may be reconstituted in the vesicle. In array format, they may thus be used to screen binding of drug candidates (e.g., enzyme inhibitor, receptor agonist or antagonist) or the functional responses induced by such binding.

### Materials & Methods

Vesicle Production

A dried lipid film was rehydrated overnight in 200 mM D-sorbitol. A vesicle cloud was then harvested (about 1 mg/ml), freeze thawed, and extruded through 100-nm filter pores (typical S.D.±40 nm). The vesicles used for the experiments in FIG. 4, after harvesting were passed once through a 1 μm pore-size filter to create a sharp upper cut-off in their size distribution. Before they were incubated on surfaces, vesicles were diluted (1:10) in the immobilization buffer: 80 mM NaCl and 10 mM NaHPO₄ at pH 7.4. All of the vesicles are composed of 88% about POPC, about 10% POPG, about 2% n-((6-(biotinoyl)amino)-hexanoyl)-1,2-dihexadecanoyl-sn-glycero-3-phosphoethanolamine (DHPE-biotin), and about 0.3% 1,2-dioleoyl-sn-glycero-3-phosphoethanolamine-n-[methoxy(polyethylene glycol)-2000] (DOPE-PEG₂₀₀₀). Two fluorescently labeled lipids were used: n-(6-tetramethylrhodaminethio-carbamoyl)-1,2-dihexadecanoyl-sn-glycero-3-phosphoethanolamine (TRITC-DHPE) and OREGON GREEN® 488-1,2-dihexadecanoyl-sn-glycero-3-phosphoethanolamine (Oregon 488-DHPE).

Patterned Surface Functionalization

Poly(dimethylsiloxane) stamps for μCP were inked with 0.1 mg/ml BSA-biotin. After washing the stamps with PBS and drying them under a N₂ stream, we printed BSA-biotin on a clean glass substrate that was subsequently blocked with 0.5 mg/ml BSA. The patterned substrates were then functionalized with 0.025 mg/ml streptavidin incubated for 10 min. The final streptavidin surface density was about 10% of a complete monolayer.

Calibration of pH Ratio for Carboxyfluoroscein

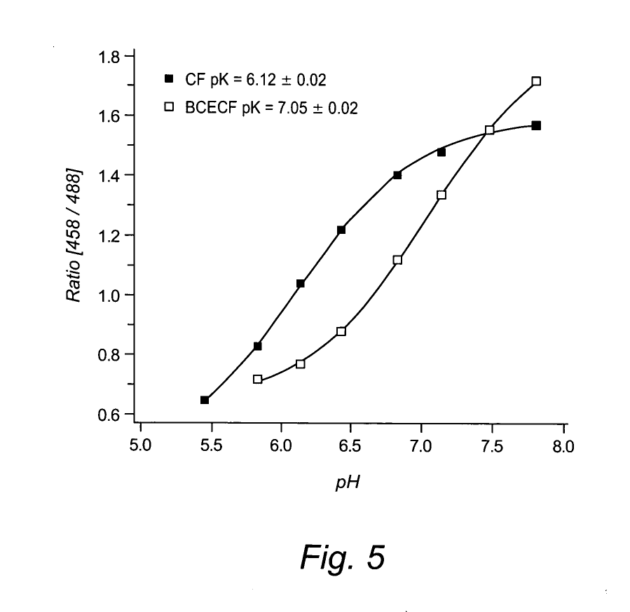

The carboxyfluorescein (CF) dye response (excitation ratio 488 nm/458 nm, emission bandwidth of 505 nm to 550 nm) was calibrated in solutions of different known pH values^{[27]} (buffer: 200 mM D-sorbitol, 10 mM NaHPO₄). The calibration curve (FIG. 5) was recorded by scanning with a confocal microscope inside the buffered CF solutions using the exact settings with which the vesicle images were acquired. For each time point in FIG. 4, two images of immobilized vesicles were recorded with 458 nm and 488 nm, respectively. The intensity of each vesicle was noted in each of the two images to calculate the corresponding ratio value for a vesicle at a given time. The 2′,7′-bis-(2-carboxyethyl)-5-(and-6)-carboxyfluorescein dye (BCECF) is a fluorescent indicator of vesicle pH with a neutral pKa (similar to the cytoplasm) and a pH-dependent excitation profile.

Insertion of Gramicidin Lipid Bilayer

The stock solution of gramicidin A was 100 μM in chloroform. At the beginning of each day, an ethanol (EOH) solution of 1 μM was prepared from the stock solution. The EOH solution was finally diluted in working buffer at a ratio of 1/100 to give a final concentration of 10 nM gramicidin A^{[28]}. The sample was mixed manually with a pipette and then equilibrated for 2 min to 3 min to assure sufficient time for insertion and dimerization of gramicidin A in the lipid bilayer^{[29]}. Injection of the same EOH quantity did not have any effect on vesicle pH.

Sodium Influx Induced by Gramicidin

To trigger the pH increase, we used the peptide gramicidin A which has a single channel conductivity^{[30]} of ˜40 pS and an initial current of ˜12 pA or 10⁸ ions/sec under our conditions. The half time of the pH response upon gramicidin A-induced ion flux in vesicles has been measured before^{[31]} but is also straightforward to approximate. Using the Nernst equation, for external and internal Na⁺ concentration of 0.1 M and 10⁻⁶ M, we can calculate the electrochemical equilibrium potential E_{Na}˜0.3 V. To create this potential difference of a vesicle which is 1 μm in diameter, assuming a membrane capacitance of 1 μF/cm², we need a net inflow of about 10⁴ ions. So at a first approximation, the half time of Na⁺/pH increase is in the order of about 10⁴ ions/10⁸ ions sec⁻¹=0.1 msec. In agreement to this rough calculation, the time response is reported^{[31]} to be <1 msec. An ion channel with single channel current of ˜1 fA would have a half time about 10⁴-fold longer (i.e., in the range of seconds).

### REFERENCES

1. Lipowsky & Sackmann (1995) Structure and Dynamics of Membranes. From Cells to Vesicles. Elsevier Science, Amsterdam.
2. Gennis (1989) Biomembranes. Molecular Structure and Function, 1^{st} Ed. Springer, New York, N.Y.
3. Bernard et al. (2001) Anal. Chem. 73, 8.
4 Berg et al. (2000) Micro Total Analysis Systems 2000. Kluwer Academic, Dordrecht.
5. Jensen (1998) Nature 393, 735.
6. Leven et al. (2003) Science 299, 682.
7. Chiu et al. (1999) Science 283, 1892.
8. Michel et al. (2001) IBM J. Res. Develop. 45, 697; Xia & Whitesides (1998) Angew. Chem. 110, 568; Angew. Chem. Int. Ed. 37, 550.
9. Whitesides & Grzybowski (2002) Science 295, 2418.
10. Yin et al. (2001) J. Am. Chem. Soc. 123, 8718.
11. Svedhem et al. (2003) Chembiochem 4, 339; Michel et al. (2002) Langmuir 18, 8580.
12. Walker et al. (1997) Nature 387, 61.
13. Albersdorfer et al. (1997) Biophys. J. 73, 245.
14. Lipowsky (1991) Nature 349, 475.
15. Kroger et al. (1999) Anal. Chem. 71, 3157.
16. Frisken et al. (2000) Langmuir 16, 928.
17. Bieri et al. (1999) Nat. Biotechnol. 17, 1105.
18. Renault et al. (2003) J. Phys. Chem. B 107, 703
19. Walt (2000) Science 287, 451.
20. Biegel & Gould (1981) Biochemistry 20, 3474.
21. Kelley et al. (2003) Nature 424, 321.
22. Tschodrich-Rotter et al. (1996) Biophys. J. 70, 723.
23. Demers et al. (2002) Science 296, 1836.
24. Rhoades et al. (2003) Proc. Natl. Acad. Sci. USA 100, 3197.
25. Chiu et al. (1998) Science 279, 1190.
26. Michel et al. (2002) Langmuir 18, 3281.
27. Mordon et al. (2002) Photochem. Photobiol. 60, 274.
28. Clement & Gould (1981) Biochemistry 20, 1544.
29. Loew et al. (1983) Biochemistry 22, 837.
30. Rostovtseva et al. (1998) Biophys. J. 75, 1783.
31. Biegel & Gould (1981) Biochemistry 20, 3474.

The invention has also been described in Stamou et al. (Agnew. Chem. Int. Ed. 42, 5580-5583, 2003); the contents of which are incorporated by reference. Patents, patent applications, and other publications cited herein are also incorporated by reference in their entirety.

In stating a numerical range, it should be understood that all values within the range are also described (e.g., one to ten also includes every integer value between one and ten as well as all intermediate ranges such as two to ten, one to five, and three to eight). The term “about” may refer to the statistical uncertainty associated with a measurement or the variability in a numerical quantity which a person skilled in the art would understand does not affect operation of the invention or its patentability.

All modifications and substitutions that come within the meaning of the claims and the range of their legal equivalents are to be embraced within their scope. A claim using the transition “comprising” allows the inclusion of other elements to be within the scope of the claim; the invention is also described by such claims using the transitional phrase “consisting essentially of” (i.e., allowing the inclusion of other elements to be within the scope of the claim if they do not materially affect operation of the invention) and the transition “consisting” (i.e., allowing only the elements listed in the claim other than impurities or inconsequential activities which are ordinarily associated with the invention) instead of the “comprising” term. Any of these three transitions can be used to claim the invention.

It should be understood that an element described in this specification should not be construed as a limitation of the claimed invention unless it is explicitly recited in the claims. Thus, the granted claims are the basis for determining the scope of legal protection instead of a limitation from the specification which is read into the claims. In contradistinction, the prior art is explicitly excluded from the invention to the extent of specific embodiments that would anticipate the claimed invention or destroy novelty.

Moreover, no particular relationship between or among limitations of a claim is intended unless such relationship is explicitly recited in the claim (e.g., the arrangement of components in a product claim or order of steps in a method claim is not a limitation of the claim unless explicitly stated to be so). All possible combinations and permutations of individual elements disclosed herein are considered to be aspects of the invention. Similarly, generalizations of the invention's description are considered to be part of the invention.

From the foregoing, it would be apparent to a person of skill in this art that the invention can be embodied in other specific forms without departing from its spirit or essential characteristics. The described embodiments should be considered only as illustrative, not restrictive, because the scope of the legal protection provided for the invention will be indicated by the appended claims rather than by this specification.

1. An array, said array comprising:
(a) a surface and receptors attached thereon in a pattern such that said receptors are located in regions of said surface, wherein receptors are not attached in areas separating each region from other regions and
(b) intact single vesicles comprised of a lipid bilayer and ligands exposed on the exterior side of said lipid bilayer;
an intact single vesicle being located at one or more region(s) on said surface by specific binding between ligand exposed on said vesicle and receptor attached to said region(s), wherein said lipid bilayer of said vesicle is comprised of charged lipids, uncharged lipids, and hydrophilic modified lipids.

2. The array of claim 1, wherein there are at least 10⁶ regions per mm² of said surface.

3. The array of claim 1, wherein the closest of said regions are separated from each other by at least a center-to-center distance from 1 μm to 10 μm.

4. The array of claim 1, wherein the closest of said regions are separated from each other by at most a center-to-center distance from 1 μm to 10 μm.

5. The array of claim 1, wherein the average diameter of said vesicles is at least from 50 nm to 500 nm.

6. The array of claim 1, wherein the average diameter of said vesicles is at most from 50 nm to 500 nm.

7. The array of claim 1, wherein there are an average from 0.5 to 10 of said vesicle(s) immobilized in each region.

8. The array of claim 1, wherein said charged lipids are at least 10 mol % of said vesicle's lipids.

9. The array of claim 1, wherein said hydrophilic modified lipids are modified with at least a poly(ethylene glycol) (PEG).

10. The array of claim 1, wherein said receptor and said ligand are streptavidin and biotin, respectively.

11. The array of claim 1, wherein at least chemical reagents or proteins are encapsulated in said vesicle's interior and/or embedded in its lipid bilayer.

12. A method of producing an array, said method comprising:
(a) attaching receptors to regions and not to areas of said array;
(b) making single intact vesicles, wherein a lipid bilayer spatially compartmentalizes each vesicle's interior and exterior, comprising charged lipids, uncharged lipids, and hydrophilic modified lipids with ligand attached to said lipid bilayer and exposed on said exterior; and
(c) immobilizing vesicles at said regions and not at said areas through specific receptor-ligand binding.

13. The method according to claim 12, wherein said receptors are attached by contact printing.

14. The method according to claim 12, wherein said vesicles are made by extrusion.

15. The method according to claim 12, wherein said charged lipids are at least 10 mol % of said vesicle's lipids.

16. The method according to claim 12, wherein said hydrophilic modified lipids are modified with at least a poly(ethylene glycol) (PEG).

17. The method according to claim 12, wherein said receptor and said ligand are streptavidin and biotin, respectively.

18. The method according to claim 12, wherein at least chemical reagents or proteins are encapsulated in said vesicle's interior and/or embedded in its lipid bilayer.

19. A method of using the array of claim 1, said method comprising:
(a) immobilizing a plurality of chemical reagents or proteins in different vesicles of said array and
(b) reacting contents of said vesicles.

20. A method of using the array of claim 1, said method comprising:
(a) immobilizing a combinatorial library of chemical compounds or enzymes contained in vesicles of said array and
(b) screening the combinatorial library for chemical reactivity or enzymatic activity.
